(19) United States
(12) Patent Application Publication
(10) Pub. No.: US 2006/0093666 A1
(43) Pub. Date: May 4, 2006

(54) Formulations for tyrosine kinase inhibitors

(76) Inventors: Shyam B. Karki, Lansdale, PA (US); Sameer R. Deshpande, Lansdale, PA (US); Karen C. Thompson, Lansdale, PA (US); Anne H. Payne, Norristown, PA (US); Thomas P. Gandek, Collegeville, PA (US)

Correspondence Address: MERCK AND CO., INC, P O BOX 2000, RAHWAY, NJ, 07065-0907, US

(21) Appl. No.: 10/544,213

(22) PCT Filed: Mar. 23, 2004
(86) PCT No.: PCT/US04/08828

§ 371 Date: Aug. 2, 2005

Related U.S. Application Data
(60) Provisional application No. 60/458,094, filed on Mar. 27, 2003.

(51) Int. Cl. A61K 9/20; A61K 31/496

(52) U.S. Cl. 424/464; 514/253.07

(57) ABSTRACT

The present invention is related to a powder, powder blend or granulation formulation of 3-[5-(4-methanesulfonyl-piperazin-1-ylmethyl)-1H-indol-2-yl]-1H-quinolin-2-one, a tyrosine kinase inhibitor, which is adapted for reconstitution with a diluent. This invention is also related to a prepared aqueous suspension, or dispersion, formulation, particularly to a stable oral pharmaceutical formulation, comprising granules of 3-[5-(4-methanesulfonyl-piperazin-1-ylmethyl)-1H-indol-2-yl]-1H-quinolin-2-one mixed with a diluent. Additionally, the present invention is related to the method of preparing these formulations.

### BACKGROUND OF THE INVENTION

Angiogenesis is characterized by excessive activity of vascular endothelial growth factor (VEGF) (as described in U.S. Pat. No. 6,245,759 B1). KDR mediates the mitogenic function of VEGF whereas Flt-1 appears to modulate non-mitogenic functions such as those associated with cellular adhesion. Inhibiting KDR thus modulates the level of mitogenic VEGF activity. In fact, tumor growth has been shown to be susceptible to the antiangiogenic effects of VEGF receptor antagonists. (Kim et al., Nature 362, pp. 841-844, 1993).

Solid tumors can be treated by tyrosine kinase inhibitors since these tumors depend on angiogenesis for the formation of the blood vessels necessary to support their growth. These solid tumors include histiocytic lymphoma, cancers of the brain, genitourinary tract, lymphatic system, stomach, larynx and lung, including lung adenocarcinoma and small cell lung cancer. Additional examples include cancers in which overexpression or activation of Raf-activating oncogenes (e.g., K-ras, erb-B) is observed. Such cancers include pancreatic and breast carcinoma. Accordingly, inhibitors of these tyrosine kinases are useful for the prevention and treatment of proliferative diseases dependent on these enzymes.

Inhibition of KDR or Flt-1 is implicated in pathological angiogenesis, and these receptors are useful in the treatment of diseases in which angiogenesis is part of the overall pathology, e.g., inflammation, diabetic retinal vascularization, as well as various forms of cancer since tumor growth is known to be dependent on angiogenesis. (Weidner et al., N. Engl. J. Med., 324, pp. 1-8, 1991).

Compounds containing a quinoline moiety, such as 3-[5-(4 methanesulfonyl-piperazin-1-ylmethyl)-1H-indol-2-yl]-1H-quinolin-2-one, have been generically and specifically disclosed in U.S. Pat. No. 6,306,874, which issued on Oct. 23, 2001.

Inhibitors of tyrosine kinase are therefore useful for treating cancer. Since young or elderly patients may have difficulty in swallowing tablets, an oral suspension containing a tyrosine kinase inhibitor may be useful.

### SUMMARY OF THE INVENTION

The present invention is related to a granulation formulation of 3-[5-(4-methanesulfonyl-piperazin-1-ylmethyl)-1H-indol-2-yl]-1H-quinolin-2-one, a tyrosine kinase inhibitor, which is adapted for reconstitution with a diluent. This invention is also related to a prepared aqueous suspension, or dispersion, formulation, particularly to a stable oral pharmaceutical formulation, comprising granules of 3-[5-(4-methanesulfonyl-piperazin-1-ylmethyl)-1H-indol-2-yl]-1H-quinolin-2-one mixed with a diluent. Additionally, the present invention is related to the method of preparing these formulations.

### BRIEF DESCRIPTION OF THE DRAWINGS

FIG. 1 illustrates a flow diagram of the method of preparing a granulation formulation of 3-[5-(4-methanesulfonyl-piperazin-1-ylmethyl)-1H-indol-2-yl]-1H-quinolin-2-one, a tyrosine kinase inhibitor, which is adapted for reconstitution with a diluent.

### DETAILED DESCRIPTION OF THE INVENTION

In a first embodiment, the instant invention is a powder formulation adapted for reconstitution with a diluent which comprises
a) 3-[5-(4-methanesulfonyl-piperazin-1-ylmethyl)-1H-indol-2-yl]-1H-quinolin-2-one, as an active ingredient, and
b) at least one filler, wherein said filler(s) are about 10% to about 75% of the weight of the powder formulation.

In a second embodiment, the instant invention is a powder blend formulation adapted for reconstitution with a diluent which comprises
a) 3-[5-(4-methanesulfonyl-piperazin-1-ylmethyl)-1H-indol-2-yl]-1H-quinolin-2-one, as an active ingredient, and
b) at least one filler, wherein said filler(s) are about 10% of the weight of the blended formulation.

In a third embodiment, the instant invention is a granulation formulation adapted for reconstitution with a diluent which comprises
a) granules of 3-[5-(4-methanesulfonyl-piperazin-1-ylmethyl)-1H-indol-2-yl]-1H-quinolin-2-one as an active ingredient;
b) at least one binder; and
c) at least one filler, wherein said filler(s) is about 10% to about 75% of the weight of the granulation formulation.

In a further embodiment of the instant invention, the formulations described above further comprise one or more pharmaceutically acceptable excipients selected from binders, disintegrants, lubricants, flavorings, sweeteners, buffering agents, stabilizers, and viscosity modifiers.

Water may also be used, in combination with the diluent, to reconstitute the powder, powder blend or granulation formulation to a suspension.

In another embodiment, the instant invention is a method of preparing a granulation formulation, as described above in the first embodiment which comprises:
a) preparing wet granules comprising 3-[5-(4-methanesulfonyl-piperazin-1-ylmethyl)-1H-indol-2-yl]-1H-quinolin-2-one and at least one filler via wet granulation;
b) drying the wet granules and then milling to produce milled granules;
c) lubricating the milled granules with a lubricant to produce the granulation formulation; and
d) filling a container with the granulation formulation.

A further embodiment of the instant invention is a kit for preparing a pharmaceutical suspension which comprises
a) 3-[5-(4-methanesulfonyl-piperazin-1-ylmethyl)-1H-indol-2-yl]-1H-quinolin-2-one;
b) a diluent; and
c) at least one filler.

Another embodiment of the instant invention is a method of preparing a pharmaceutical supsension of 3-[5-(4-methanesulfonyl-piperazin-1-ylmethyl)-1H-indol-2-yl]-1H-quinolin-2-one, which comprises mixing a granulation formulation comprising 3-[5-(4-methanesulfonyl-piperazin-1-ylmethyl)-1H-indol-2-yl]-1H-quinolin-2-one and at least one filler, with a diluent.

Water may also be used, in combination with the diluent, to reconstitute the granulation formulation to a suspension.

In a fourth embodiment, the instant invention is an aqueous suspension formulation which comprises granules of 3-[5-(4-methanesulfonyl-piperazin-1-ylmethyl)-1H-indol-2-yl]-1H-quinolin-2-one mixed with a diluent.

A fifth embodiment of the instant invention is a method of treating cancer in a pediatric or adult patient comprising administering to a patient in need thereof an effective amount of a granulation formulation.

A sixth embodiment of the instant invention is a method of treating cancer in a pediatric or adult patient comprising administering to a patient in need thereof an effective amount of an aqueous suspension formulation.

The preparation of 3-[5-(4-methanesulfonyl-piperazin-1-ylmethyl)-1H-indol-2-yl]-1H-quinolin-2-one is described in U.S. Pat. No. 6,306,874, which issued on Oct. 23, 2001 and is herein incorporated by reference in its entirety. In addition, 3-[5-(4-methanesulfonyl-piperazin-1-ylmethyl)-1H-indol-2-yl]-1H-quinolin-2-one may also be prepared by utilizing the process described in U.S. 2002-0198252, which published on Dec. 26, 2002.

Formulations in accordance with this invention provide a powder, powder blend or granulation of 3-[5-(4-methanesulfonyl-piperazin-1-ylmethyl)-1H-indol-2-yl]-1H-quinolin-2-one for reconstitution as a suspension for oral administration. The present formulation may be packaged as a suspension or the components of the formulation may be packaged separately in a kit, which is delivered to the appropriate user, such as a doctor or a hospital pharmacy. Once delivered, the components may be reconstituted as a suspension, as described herein, and administered to a person in need. For example, at a clinical site, the appropriate user would add 5 mL of purified water to a container, such as a PET bottle, containing 4 g of 3-[5-(4-methanesulfonyl-piperazin-1-ylmethyl)-1H-indol-2-yl]-1H-quinolin-2-one granules and shake gently. Then 95 mL of simple syrup would be added and the container would be shaken. Prior to dosing a patient, the suspension would be shaken again.

As used herein, a powder blend is a mixture of two or more powders. As used herein, granules refers to agglomerates of particles bound together by a binder, which improves the flow of the powder.

Examples of the diluent which may be utilized in the instant invention include, but are not limited to, Humco's Simple Syrup, Emerson Cherry Syrup, Paddock's Ora-Sweet® Syrup, Paddock's Ora-Plus® Oral Suspending Vehicle, Ora-Sweet SF™ Sugar Free Syrup, combinations of the described diluents, and the like. Additionally, the diluent may be mixture of water and a powder such as Acacia Powder, Humco's Dextrose Powder, and the like. In a specific embodiment of the instant invention, Humco's Simple Syrup is utilized as the diluent.

Examples of fillers utilized in the instant invention include, but are not limited to, one or more of microcrystalline cellulose, lactose hydrous, dipac, mannitol, dextrose, sucrose, dibasic calcium phosphate, tribasic calcium phosphate and the like. In a specific embodiment, the filler is microcrystalline cellulose or lactose hydrous.

Examples of binders that may be utilized in the instant invention include, but are not limited to, hydroxypropyl cellulose EXF (HPC-EXF), other grades of BPC (such as BPC, HPC-SL), hydroxypropylmethyl cellulose (HNPC), starch 1500, polyvinylpyrrolidinone (PVP), hydrogenated vegetable oil, and the like. In a specific embodiment of the instant invention, HPC-EXF is used. Examples of disintegrants include but are not limited to croscarmellose sodium, povidone, crospovidone, starch 1500, sodium starch glycolate and the like. In a specific embodiment of the instant invention, the disintegrant is croscarmellose sodium.

Examples of lubricants that may be utilized in the instant invention include, but are not limited to, magnesium stearate, stearic acid, talc powder, and the like. Examples of buffering agents that may be utilized in the instant invention include, but are not limited to, citric acid, benzoic acid, acetic acid, ascorbic acid, tartaric acid, maleic acid, malic acid, lactic acid, succinic acid, phosphoric acid, fumaric acid, and the like. Examples of stabilizers that may be utilized in the instant invention include, but are not limited to, HPC, HPC-SL, HPMC, methyl cellulose, hydroxypropyl cellulose, ethyl cellulose, surfacants, and the like.

Examples of viscosity modifiers that may be utilized in the instant invention include, but are not limited to, HPC, HPMC, xanthan gum, polydextrose, sucrose, gelatin and the like.

As noted above, granules are agglomerates of particles bound together by a binder, which improves the flow of the powder. Granulation is the process for making granules and may be a wet or dry process. As understood in the art, a dry granulation process utilizes roller compaction, while wet granulation utilizes a liquid, e.g. a solvent, to granulate. In the instant invention, the granules of the granulation formulation are prepared via wet granulation in a high shear granulator. These granules comprise the HCl salt of 3-[5-(4-methanesulfonyl-piperazin-1-ylmethyl)-1H-indol-2-yl]-1H-quinolin-2-one, microcrystalline cellulose, a filler such as lactose hydrous, a binder such as hydroxypropyl cellulose EXF (HPC-EXE) and a disintegrant, such as croscarmellose sodium. Water may be used as the granulating solvent. The wet granules are dried in a fluid-bed dryer and are then milled in a Comil. The milled granules are lubricated, with a lubricant such as magnesium stearate, and filled into a container. In a specific embodiment of the instant invention, the container used is a Polyethylene Terephthalate (PET) bottle.

Generally other types of celluloses which have greater swelling ability, may be used for preparation of suspensions in lower concentrations (0.2 to 5%) acting as viscosity-increasing agent (thickener). Microcrystalline cellulose is used primarily as a diluent in oral tablet and capsule formulations.

Microcrystalline cellulose with a particle size from 20 to 100 μm is preferred. Suitable grades include Avicel types pH 101, 102, 103, 104, 112, 113, 301 and 302. These differ in physical characteristics such as particle size, bulk density, loss on drying, viscosity and chemical characteristics such as the degree of polymerization.

The percentages or amounts referred to in this specification are by weight unless indicated otherwise. Percentages or proportions are selected to total 100%.

In the formulations of this invention, predried cellulose used as a filler acting simultaneously as a viscosity-increasing agent and a stabilizing agent provides the good stability of the reconstituted suspension over the period of use. The amount of cellulose, as a principal filler in the formulation, may range from about 5 to about 90% w/w. In specific embodiment of the instant invention, the range is about 10 to about 75% w/w. In a further embodiment of the instant invention, the range is about 10 to about 70% w/w of the dry formulation. The percentage of the active substances is from about 1 to about 90%. In specific embodiment of the instant invention, the percentage of the active substances is from about 1 to about 70%. In a further embodiment, the percentage of the active substances is from about 1 to about 50%. Additional excipients may be present in the present invention, in various amounts, such that the percentages or proportions of the ingredients of the present formulation total 100%.

Microcrystalline cellulose (Avicel, Emcocel, Vitacel) with an average particle size of 20 μm or preferably microcrystalline cellulose of average particle size of 50 μm may be used. Powdered cellulose (Vivacel, Elcema, SolkaFlok) having different particle size or as granulated powder may be used. The formulations of this invention may also contain auxiliary ingredients which may be essentially conventional in the art. To improve the taste, flavorings and sweetening agents, preferably saccharin, saccharin sodium or aspartame in the amounts allowable for oral formulations may be added. Flavorings which may be used may comprise common flavors like strawberry, cherry, wild cherry, lemon, banana, raspberry, orange, caramel or mixtures thereof, which in combination with the antibiotic provide a pleasant flavor and taste.

Suitable excipients may include buffering agents such as different acids and their salts, eg citric acid, sodium citrate, succinic acid, swelling agents and viscosity-increasing agents such as suspension stabilizers and other additives.

The formulations of present invention are suitable for BID or TID administration in the prescribed dose. They are indicated in the treatment of children, adults and the elderly, and patients with difficulty in swallowing.

Formulations of the instant invention may be stored in air-tight screwcap bottles or plastic containers or in sachets for preparation of suspension or dispersion, respectively, immediately prior to use.

The formulations of the present invention can be produced using the conventional manufacturing procedures such as homogenization, sieving and milling. A portion of the ingredients may be pre-granulated, or granulated ingredients are used to improve powder flowability, which is especially important for sachet packaging.

### EXAMPLES

Examples provided are intended to assist in a further understanding of the invention. Particular materials employed, species and conditions are intended to be further illustrative of the invention and not limiting of the reasonable scope thereof.

### Example 1

### Tert-butyl 5-{[4-tert-(butoxycarbonyl)piperazin-1-yl]methyl}-1H-indole-1-carboxylate 1-4

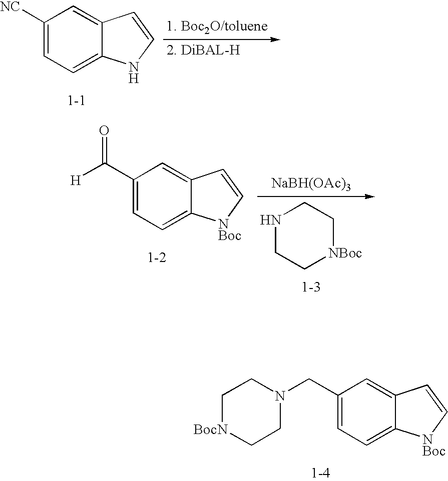

To a 50 L round bottomed flask was added toluene (8 L), 5-cyanoindole 1-1 (2 Kg, 1 eq.), and 4-(dimethylamino) pyridine (DMAP) (17 g, 0.01 eq.). Boc₂O (3.15 Kg, 1.03 eq) was then added slowly as a solution in toluene (2 L), maintaining a temperature of about 20° C. to about 30° C. Tetrahydrofuran (1) (8 L) was then added as a flush. After 30 minutes, the mixture was assayed using the HPLC assay described below and then cod to a temperature of about 15° C. to about 18° C. diisobutylalumninum hydride (DiBAL) (21.5 L; 1.5 M in toluene; 2.3 eq.) was added over 3 hours, maintaining the temperature at about 15° C. to about 18° C. The solution was aged at room temperature for one hour to overnight, and then assayed by HPLC. Additional DiBAL (˜1 L) may be added to bring the assay of Boc-cyanoindole below 1 mol %.

The DiBAL reaction mixture was charged into half of an aqueous solution of NaHSO₄ (20 Kg) in water (60 L) while maintaining the temperature at about 35° C. to about 45° C. The rate of addition was governed by the ability to maintain the temperature at about 35° C. to about 45° C., and control the amount of gas evolution.

The aqueous phase was cut at about 35° C. to about 45° C. and the remaining bisulfate solution was charged to the organic phase. After a 15 minutes at 35° C. to about 45° C., the aqueous phase was cut and the organic phase was washed with water (8 L) and brine (8 L) before being transferred to carboys containing about 5 to about 10 Kg of Na₂SO₄ to remove second phase water. A small amount of red oil, residual over-reduced byproduct, appeared at the interface of the aqueous cuts, and was cut forward with the aqueous.

A 100 L extractor was washed with water and dried via THF boil-out, then the organic phase was recharged though a 10 micron line filter, followed by a toluene rinse (4 L). Boc-piperazine 1-3 (2.61 Kg, 1 eq) was added, then sodium triacetoxyborohydride (3.86 Kg, 1.3 eq) was added in portions while maintaining the temperature from about 23° C. to about 27° C. This addition was moderately exothermic. The mixture was aged for 1.5 hours, assayed and then quenched by adding 2.5 v/v % acetic acid in water (20 L). The total volume after quenching was about 80 L.

The organic phase was washed with water (20 L), the aqueous phase was cut and the organic phase solvent was switched to MeOH via in vacuo batch concentration in the 50 L round bottom to a target volume of 25 L. The batch was warmed to a temperature of about 30° C. to about 35° C. and seeded. After a good seed bed had formed, 60/40 water/methanol (20 L) was added over 1 hour and the batch chilled to about 5° C. and aged for 1 hour. The product was isolated via filtration, washed (3 L, 70:30 MeOH/water) and dried via a nitrogen purge. About 5 Kg (85%) of tert-butyl 5-{[4-tert-(butoxycarbonyl) piperazin-1-yl]methyl}-1H-indole-1-carboxylate 1-4 was obtained as a white solid.

### Example 2

### Preparation of Boronic Acid Intermediate 2-1

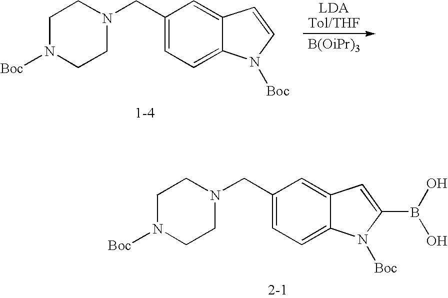

A mixture of 1-4 (2780 g; 6.69 mol), 11.1 L of toluene and 2.8 L of ThP (tetrahydrofuran) was cooled to −78° C. 5.4 L (10.7 mol) of 2M LDA (lithium diisopropylamide) was then added slowly so as to keep the temperature below about −70° C. The reaction mixture was then aged for two hours.

4.6 L (19.9 mol) of triisopropylborate was added slowly while maintaining the temperature below about −70° C. The reaction is done when the remaining amount of 1-4 is two percent or less. Additional LDA may be added if necessary to drive the reaction to completion. After 30 minutes, the reaction was warmed to about 0° C. with an ice bath. The reaction was then quenched with 12 L of 2N HCl (24.1 mol) and the pH adjusted to about 7. The ice bath was removed and the biphasic solution was stirred for about 30 minutes to ensure that everything was in solution. The layers were then separated and the organic layer was used in the next reaction without further purification.

### Example 3

### Preparation of Quinindole Intermediate 3-2

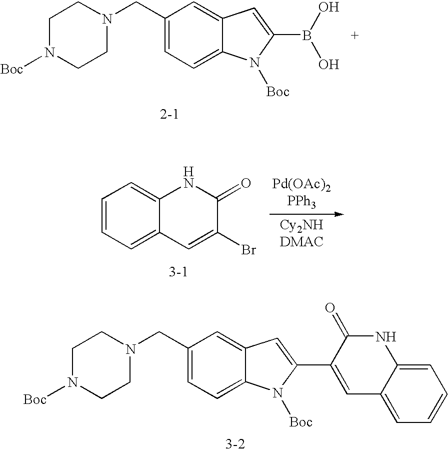

In a 50 L round bottom flask was combined the 3-bromoquinolin-2-one (1 kg, 4.46 moles), palladium acetate (50.1 g, 0.223 moles), PPh₃ (117 g, 0.446 moles), dicyclohexylamine (2.7 L, 13.4 moles) and dimethylacetamide (DMAC) (10 L). The solution was degassed two times and purged with nitrogen each time. The reaction mixture was heated to 60° C. At 60° C. the boronic acid (prepared as described in Example 2) (3.073 kg, 6.69 moles) was then added (this solution is not degassed) as a solution over a two hours period. The reaction was then aged overnight.

The reaction is assayed by HPLC. The reaction is done after the disappearance of either quinolinone or boronic acid. The ratio of desired product to undesired should be 3.5:1 or better.

Darco KB (125 g; 5 wt % of theory yield) was added to the reaction mixture. The mixture was heated at 60° C. for 30 min then cooled to room temperature.

Celite (125 g; 5 Wt % of theory yield) was added to the reaction mixtue. The reaction was filtered and flask is rinsed with 1-2 L of toluene. The cake was then washed with 1-2 L of toluene.

The filtrate was transferred into a 100 L cylindrical extractor and warmed to 55° C. Water (10 L) was added slowly so as to maintain the temperature. The mixture was stirred for 30 minutes, then the layers were separated.

The organic layer was transferred to a 50 L round bottom flask and concentrated to a volume of 12 L or less. To the resulting mixture was added EtOAc (12 L). Stirred for at least two hours or overnight.

The resulting solids were filtered and the cake washed with a 1:1 mixture of EtOAc/Toluene (1.3 L). The solids were then dried.

### Example 4

### Deprotection of 3-2

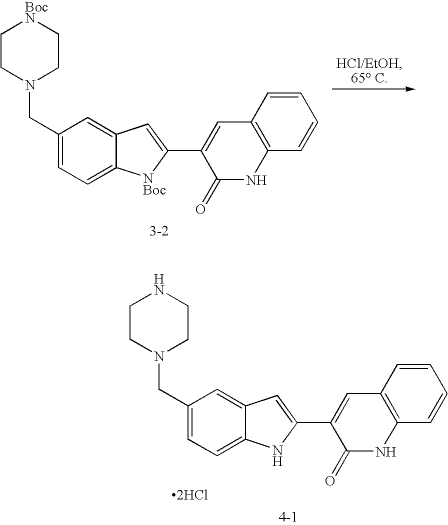

A slurry of quinindole 3-2, prepared as described in Example 3, (1.85 Kg) in absolute ethanol (28 L) was treated with concentrated aq HCl (3.7 L) in a SOL flask. The solution was heated to 65° C. for 8 hours or more, then cooled to room temperature. The secondary amine as the bis-HCl salt was collected by filtration, with a 5 L ethanol wash.

### Example 5

### Methylsulfonation of Intermediate 4-1

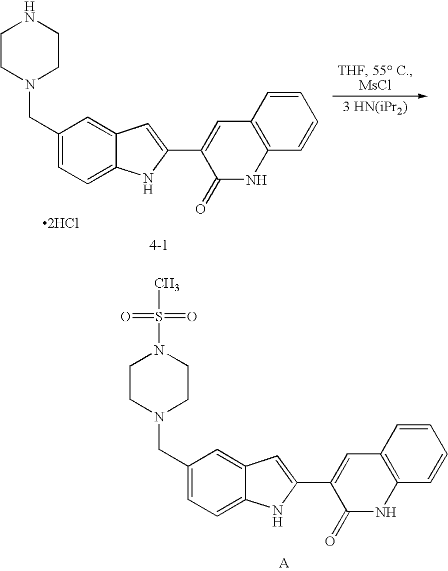

Intermediate 4-1 (1.2 kg, 2.78 moles), ThF (24 L) and diisopropylamine (1.17 L, 8.35 moles) were charged to a SOL round bottomed flask, and the slurry was heated to 55° C. Methanesulfonyl chloride was added over 3 hours, and the thick yellow slurry was stirred 4 hours or overnight. The mixture was cooled to room temperature, then water (15.6 L) was charged over 1.5 hours. To the last five liters of water was added 600 mL conc ammonium hydroxide to adjust the slurry pH to >7. (Total volume 43 L.) The slurry was aged one hour, then filtered, with a 3.6 L cake wash (60:40 THF: water). The final product was dried at 70° C. at 40 torr for several days, to provide compound A as a yellow solid.

Alternatively, the reaction can be quenched with 24 L total water.

### Example 6

### Preparation of Granulation Formulation of 3-[5-(4-methanesulfonyl-piperazin-1-ylmethyl)-1H-indol-2-yl]-1H-quinolin-2-one (Compound A)

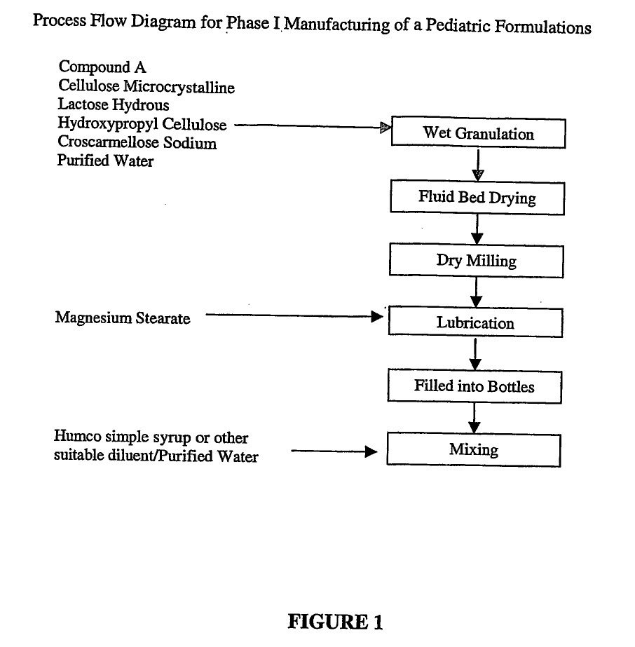

Two active formulations are prepared: 10-mg/ml and 1-mg/ml. For the 10-mg/ml formulation, 4-g of granules containing 1-g of drug are filled into PET bottles. For the 1-mg/ml formulation, 400-mg of granules containing 100-mg of drug are filled into PET bottles. At the clinical site, 100-ml of Humco simple syrup solution containing 95-ml of Humco Simple Syrup and 5-ml of water is added to the bottles to attain concentrations of 10-mg/ml and 1-mg/ml respectively. The compositions of the formulations are shown in Table 1. Flow Diagram of the manufacturing process is shown in FIG. 1. The batch containing 4-g granules/bottle was placed on stability according to protocols described.

TABLE 1
Composition of 3-[5-(4-methanesulfonyl-piperazin-1-ylmethyl)-
1H-indol-2-yl]-1H-quinolin-2-one Granulation Formulations

| | Unit Strength | |
|---|---|---|
| | 1 g | 100 mg |
| Ingredient | mg/bottle | mg/bottle |
| Core Tablet | | |
| Compound A HCl salt* | 1080.0 | 108.0 |
| (as free base) | (1000.0) | (100.0) |
| Cellulose Microcrystalline NF(Avicel PH101) | 800.0 | 80.0 |
| Lactose Hydrous NF | 1860.0 | 186.0 |
| Hydroxypropyl Cellulose (Klucel-EXF) NF | 120.0 | 12.0 |
| Croscarmellose Sodium NF (Ac-Di-Sol) | 120.0 | 12.0 |
| Magnesium Stearate NF (Non-Bovine) | 20.0 | 2.0 |
| Purified Water** USP | — | — |
| Total weight of granules in bottles | 4000 | 400 |

*1 mg of Free Base = 1.08 mg of HCl salt
**removed during processing

What is claimed is:

1. A powder formulation adapted for reconstitution with a diluent which comprises
a) 3-[5-(4-methanesulfonyl-piperazin-1-ylmethyl)-1H-indol-2-yl]-1H-quinolin-2-one, as an active ingredient, and
b) at least one filler, wherein said filler(s) are about 10% to about 75% of the weight of the powder formulation.

2. The powder formulation of claim 1, wherein the filler is selected from microcrystalline cellulose, lactose hydrous, Dipac, Mannitol, and a combination thereof.

3. The powder formulation of claim 2, wherein the filler is microcrystalline cellulose, lactose hydrous or a combination thereof.

4. A powder blend formulation adapted for reconstitution with a diluent which comprises
a) 3-[5-(4-methanesulfonyl-piperazin-1-ylmethyl)-1H-indol-2-yl]-1H-quinolin-2-one, as an active ingredient, and
b) at least one filler, wherein said filler(s) are about 10% of the weight of the blended formulation.

5. The powder blend formulation of claim 4, wherein the filler is selected from microcrystalline cellulose, lactose hydrous, Dipac, Mannitol, and a combination thereof.

6. The powder blend formulation of claim 5, wherein the filler is microcrystalline cellulose, lactose hydrous or a combination thereof.

7. A granulation formulation adapted for reconstitution ith a diluent which comprises
a) 3-[5-(4-methanesulfonyl-piperazin-1-ylmethyl)-1H-indol-2-yl]-1H-quinolin-2-one as an active ingredient;
b) at least one binder; and
c) at least one filler, wherein said filler(s) is about 10% to about 75% of the weight of the granulation formulation.

8. The granulation formulation of claim 7, wherein the filler is selected from microcrystalline cellulose, lactose hydrous, Dipac, Mannitol, and a combination thereof.

9. The granulation formulation of claim 8, wherein the filler is microcrystalline cellulose, lactose hydrous or a combination thereof.

10. The granulation formulation of claim 7, wherein water is used in combination with a diluent for reconstitution of the granulation formulation.

11. The granulation formulation of claim 7, wherein the granulation formulation further comprises one or more pharmaceutically acceptable excipients selected from binders, disintegrants, lubricants, flavorings, sweeteners, buffering agents, stabilizers, and viscosity modifiers.

12. A method of preparing the granulation formulation of claim 7 which comprises:
a) preparing wet granules comprising 3-[5-(4-methanesulfonyl-piperazin-1-ylmethyl)-1H-indol-2-yl]-1H-quinolin-2-one and fillers via wet granulation;
b) drying the wet granules and then milling to produce milled granules;
c) lubricating the milled granules with a lubricant to produce the granulation formulation; and
d) filling a container with the granulation formulation.

13. A kit for preparing a pharmaceutical suspension which comprises
a) granules of 3-[5-(4-methanesulfonyl-piperazin-1-ylmethyl)-1H-indol-2-yl]-1H-quinolin-2-one;
b) a diluent; and
c) at least one filler.

14. The kit of claim 13, wherein the diluent is selected from Humco's Simple Syrup, Emerson Cherry Syrup, Paddock's Ora-Sweet® Syrup, Paddock's Ora-Plus® Oral Suspending Vehicle, Ora-Sweet SF™ Sugar Free Syrup, and a combination thereof.

15. The kit of claim 14 wherein the diluent is Humco's Simple Syrup.

16. The kit of claim 13, wherein the filler is selected from microcrystalline cellulose, lactose hydrous, or a combination thereof.

17. An aqueous suspension formulation which comprises granules of 3-[5-(4-methanesulfonyl-piperazin-1-ylmethyl)-1H-indol-2-yl]-1H-quinolin-2-one, at least one binder and at least one filler, mixed with a diluent.

18. The aqueous suspension formulation of claim 17, wherein the diluent is selected from Humco's Simple Syrup, Emerson Cherry Syrup, Paddock's Ora-Sweet® Syrup, Paddock's Ora-Plus® Oral Suspending Vehicle, Ora-Sweet SF™ Sugar Free Syrup, and a combination thereof.

19. The powder formulation of claim 18 wherein the diluent is Humco's Simple Syrup.

20. The aqueous suspension formulation of claim 17 which comprises granules which comprise 3-[5-(4-methanesulfonyl-piperazin-1-ylmethyl)-1H-indol-2-yl]-1H-quinolin-2-one HCl, microcrystalline cellulose, lactose hydrous, hydroxypropyl cellulose EXF and croscarmellose sodium, which are mixed with a solution of Humco Simple Syrup and water.

21. A method of preparing a pharmaceutical supsension of 3-[5-(4-methanesulfonyl-piperazin-1-ylmethyl)-1H-indol-2-yl]-1H-quinolin-2-one which comprises mixing the granulation formulation of claim 7 with a diluent.

22. The method of claim 21, wherein the diluent is selected from Humco's Simple Syrup, Emerson Cherry Syrup, Paddock's Ora-Sweet® Syrup, Paddock's Ora-Plus® Oral Suspending Vehicle, Ora-Sweet SF™ Sugar Free Syrup, and a combination thereof.

23. The method of claim 22 wherein the diluent is Humco's Simple Syrup.

24. The method of claim 21 wherein the granulation formulation is mixed with a solution of Humco's Simple Syrup and water.

25. A method of treating cancer in a pediatric or adult patient comprising administering to a patient in need thereof an effective amount of the formulation of claim 7.

26. A method of treating cancer in a pediatric or adult patient comprising administering to a patient in need thereof an effective amount of the formulation of claim 17.
